(19) United States
(12) Patent Application Publication
(10) Pub. No.: US 2024/0108250 A1
(43) Pub. Date: Apr. 4, 2024

(54) SYSTEM AND METHODS FOR IDENTIFYING TURNS

(71) Applicant: Veristride, Inc., South Salt Lake, UT (US)

(72) Inventors: Stacy J. Morris Bamberg, Salt Lake City, UT (US); Nate Godfrey, Murray, UT (US)

(21) Appl. No.: 18/540,818

(22) Filed: Dec. 14, 2023

Related U.S. Application Data
(63) Continuation of application No. 17/833,728, filed on Jun. 6, 2022.
Application No. 17/833,728 is a continuation of application No. 16/684,424, filed on Nov. 14, 2019, now abandoned.
(60) Provisional application No. 62/767,975, filed on Nov. 15, 2018.

(51) Int. Cl. A61B 5/11; A61B 5/00

(52) U.S. Cl. CPC A61B 5/112; A61B 5/1121; A61B 5/1126; A61B 5/6807; A61B 5/7282; A61B 2503/10; A61B 2505/09; A61B 2562/0219; A61B 2562/0223

(57) ABSTRACT

Determining whether an individual has turned during ambulation includes identifying a first foot parameter for the individual in at least two or more successive stances and identifying a change in the first foot parameter between the two or more successive stances. The determination also includes identifying a second foot parameter for the individual in at least two or more successive strides and identifying a change in the second foot parameter between the two or more successive strides. Based on the identified changes in the first and second foot parameters, a determination can be made whether the individual has turned during ambulation.

This application is a continuation of U.S. application Ser. No. 17/833,728, filed on Jun. 6, 2022, entitled SYSTEMS AND METHODS FOR IDENTIFYING TURNS, which is a continuation of U.S. application Ser. No. 16/684,424, filed Nov. 14, 2019, entitled SYSTEMS AND METHODS FOR IDENTIFYING TURNS, which claims priority to and the benefit of U.S. Provisional Application No. 62/767,975, filed Nov. 15, 2018, entitled SYSTEMS AND METHODS FOR IDENTIFYING TURNS, the entire contents of each of the foregoing are incorporated herein by reference.

### BACKGROUND

Computer systems and related technology affect many aspects of society. Indeed, the computer system's ability to process information has transformed the way we live and work. Computer systems now commonly perform a host of tasks (e.g., word processing, scheduling, accounting, etc.) that prior to the advent of the computer system were performed manually. More recently, computer systems have been coupled to one another and to other electronic devices to form both wired and wireless computer networks over which the computer systems and other electronic devices can transfer electronic data. In addition, computing systems (including sensors) are now often being coupled to humans to allow for performing a variety of tasks. For instance, computer systems are now being used for identification of individuals (e.g., biometrics, including finger print scanners, retina scanners, facial recognition, and so forth), for health monitoring (e.g., heart rate sensors, pulse oximeters, and so forth).

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

At least some embodiments described herein relate to determining whether an individual has turned during ambulation. For example, embodiments may include identifying a first foot parameter for the individual in at least two or more successive stances and identifying a change in the first foot parameter between the two or more successive stances. Embodiments may further include identifying a second foot parameter for the individual in at least two or more successive strides and identifying a change in the second foot parameter between the two or more successive strides. Based on the identified changes in the first and second foot parameters, a determination can be made whether the individual has turned during ambulation.

In this way, one or more components for identifying turns during ambulation may be coupled to an individual via footwear (e.g., shoes, boots, socks, and so forth) of the individual. The one or more components and potentially, one or more components separate from the footwear, may gather and analyze data associated with one or both feet of the individual, identify changes in the data, and determine if the individual has turned during ambulation. Such determinations may then allow for a physician or therapist to determine if the individual has following a training or rehabilitation program or if preventative and/or corrective measures should be taken.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an example computer architecture that facilitates operation of the principles described herein.

FIG. 2 illustrates an example environment for identifying turns during an individual's ambulation.

FIG. 3 illustrates an example of spatial differences associated with a series of steps of an individual.

FIG. 4 illustrates another example of spatial differences associated with a series of steps of an individual.

FIG. 5 illustrates another example of spatial differences associated with a series of steps of an individual.

FIG. 6 illustrates a flowchart of a method for identifying turns during ambulation of an individual.

### DETAILED DESCRIPTION

At least some embodiments described herein relate to determining whether an individual has made a turn during ambulation (e.g., walking, running, etc.). For example, embodiments may include identifying a first foot parameter for the individual in at least two or more successive stances and identifying a change in the first foot parameter between the two or more successive stances. Embodiments may further include identifying a second foot parameter for the individual in at least two or more successive strides and identifying a change in the second foot parameter between the two or more successive strides. Based on the identified changes in the first and second foot parameters, a determination can be made whether the individual has turned during ambulation.

In this way, one or more components for identifying turns during ambulation may be coupled to an individual via footwear (e.g., shoes, boots, socks, and so forth) of the individual. The one or more components and potentially, one or more components separate from the footwear, may gather and analyze data associated with one or both feet of the individual, identify changes in the data, and determine if the individual has turned during ambulation. Such determinations may then allow for a physician or therapist to determine if the individual has following a training or rehabilitation program or if preventative and/or corrective measures should be taken.

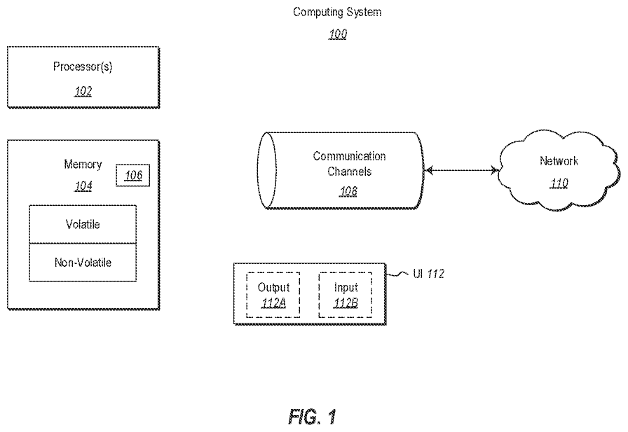

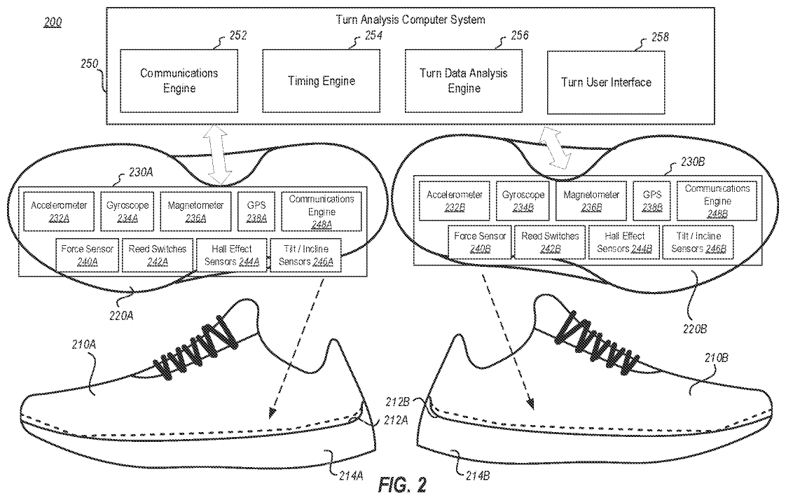

Some introductory discussion of a computing system will be described with respect to FIG. 1. Then determining whether an individual has made a turn during ambulation will be described with respect to FIGS. 2 through 6.

Computing systems are now increasingly taking a wide variety of forms. Computing systems may, for example, be handheld devices, appliances, laptop computers, desktop computers, mainframes, distributed computing systems, datacenters, or even devices that have not conventionally been considered a computing system, such as wearables (e.g., glasses). In this description and in the claims, the term “computing system” is defined broadly as including any device or system (or combination thereof) that includes at least one physical and tangible processor, and a physical and tangible memory capable of having thereon computer-executable instructions that may be executed by a processor. The memory may take any form and may depend on the nature and form of the computing system. A computing system may be distributed over a network environment and may include multiple constituent computing systems.

As illustrated in FIG. 1, in its most basic configuration, a computing system 100 typically includes at least one hardware processing unit 102 and memory 104. The memory 104 may be physical system memory, which may be volatile, non-volatile, or some combination of the two. The term “memory” may also be used herein to refer to non-volatile mass storage such as physical storage media. If the computing system is distributed, the processing, memory and/or storage capability may be distributed as well.

The computing system 100 also has thereon multiple structures often referred to as an “executable component”. For instance, the memory 104 of the computing system 100 is illustrated as including executable component 106. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed on the computing system, whether such an executable component exists in the heap of a computing system, or whether the executable component exists on computer-readable storage media.

In such a case, one of ordinary skill in the art will recognize that the structure of the executable component exists on a computer-readable medium such that, when interpreted by one or more processors of a computing system (e.g., by a processor thread), the computing system is caused to perform a function. Such structure may be computer-readable directly by the processors (as is the case if the executable component were binary). Alternatively, the structure may be structured to be interpretable and/or compiled (whether in a single stage or in multiple stages) so as to generate such binary that is directly interpretable by the processors. Such an understanding of example structures of an executable component is well within the understanding of one of ordinary skill in the art of computing when using the term “executable component”.

The term “executable component” is also well understood by one of ordinary skill as including structures that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. In this description, the terms “component”, “service”, “engine”, “module”, “control”, or the like may also be used. As used in this description and in the case, these terms (whether expressed with or without a modifying clause) are also intended to be synonymous with the term “executable component”, and thus also have a structure that is well understood by those of ordinary skill in the art of computing.

In the description that follows, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied on one or more computer-readable media that form a computer program product. An example of such an operation involves the manipulation of data.

The computer-executable instructions (and the manipulated data) may be stored in the memory 104 of the computing system 100. Computing system 100 may also contain communication channels 108 that allow the computing system 100 to communicate with other computing systems over, for example, network 110.

While not all computing systems require a user interface, in some embodiments, the computing system 100 includes a user interface 112 for use in interfacing with a user. The user interface 112 may include output mechanisms 112A as well as input mechanisms 112B. The principles described herein are not limited to the precise output mechanisms 112A or input mechanisms 112B as such will depend on the nature of the device. However, output mechanisms 112A might include, for instance, speakers, displays, tactile output, holograms, and so forth. Examples of input mechanisms 112B might include, for instance, microphones, touchscreens, holograms, cameras, keyboards, mouse of other pointer input, sensors of any type, and so forth.

Embodiments described herein may comprise or utilize a special purpose or general-purpose computing system including computer hardware, such as, for example, one or more processors and system memory, as discussed in greater detail below. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: storage media and transmission media.

Computer-readable storage media includes RAM, ROM, EEPROM, CD-ROM or other optical disk storage, magnetic disk storage or other magnetic storage devices, or any other physical and tangible storage medium which can be used to store desired program code means in the form of computer-executable instructions or data structures and which can be accessed by a general purpose or special purpose computing system.

A “network” is defined as one or more data links that enable the transport of electronic data between computing systems and/or modules and/or other electronic devices. When information is transferred or provided over a network or another communications connection (either hardwired, wireless, or a combination of hardwired or wireless) to a computing system, the computing system properly views the connection as a transmission medium. Transmissions media can include a network and/or data links which can be used to carry desired program code means in the form of computer-executable instructions or data structures and which can be accessed by a general purpose or special purpose computing system. Combinations of the above should also be included within the scope of computer-readable media.

Further, upon reaching various computing system components, program code means in the form of computer-executable instructions or data structures can be transferred automatically from transmission media to storage media (or vice versa). For example, computer-executable instructions or data structures received over a network or data link can be buffered in RAM within a network interface module (e.g., a “NIC”), and then eventually transferred to computing system RAM and/or to less volatile storage media at a computing system. Thus, it should be understood that storage media can be included in computing system components that also (or even primarily) utilize transmission media.

Computer-executable instructions comprise, for example, instructions and data which, when executed at a processor, cause a general purpose computing system, special purpose computing system, or special purpose processing device to perform a certain function or group of functions. Alternatively, or in addition, the computer-executable instructions may configure the computing system to perform a certain function or group of functions. The computer executable instructions may be, for example, binaries or even instructions that undergo some translation (such as compilation) before direct execution by the processors, such as intermediate format instructions such as assembly language, or even source code.

Those skilled in the art will appreciate that the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables (such as glasses) and the like. The invention may also be practiced in distributed system environments where local and remote computing systems, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

Computer systems and related technology, as described herein, now affect many aspects of society. Indeed, the computer system's ability to process information has transformed the way we live and work. Computer systems now commonly perform a host of tasks (e.g., word processing, scheduling, accounting, etc.) that prior to the advent of the computer system were performed manually. More recently, computer systems have been coupled to one another and to other electronic devices to form both wired and wireless computer networks over which the computer systems and other electronic devices can transfer electronic data.

In addition, computing systems (including sensors) are often being coupled to humans to allow for performing a variety of tasks. For instance, computer systems are now being used for identification of individuals (e.g., biometrics, including finger print scanners, retina scanners, facial recognition, and so forth), for health monitoring (e.g., heart rate sensors, pulse oximeters, and so forth), and a variety of other users. Such technology can be instrumental in identifying and/or preventing serious medical issues from occurring or worsening.

During physical therapy, rehabilitation therapy, or other situations, it can be desirable to monitor various aspects of an individual's ambulation. For instance, it may be desirable to monitor various aspects of an individual's ambulation to determine if the individual has made a turn during ambulation. Furthermore, it may be desirable to determine various aspects about any detected turn, including the direction of the turn, the sharpness or angle of the turn (e.g., slight veer, 45° turn, 90° turn, etc.).

Accordingly, FIG. 2 illustrates an environment 200 for determining whether an individual has turned during ambulation, which can then be used to identify physical limitations or impairments and take corrective/preventative measure to mitigate issues resulting therefrom. As illustrated, the environment 200 includes a left shoe 210A, a right shoe 210B, a left insole 220A, a right insole 220B, and a turn analysis computer system 250. The shoes 210 (i.e., the left shoe 210A and the right shoe 210B) may comprise any type of applicable footwear that can be worn on a natural foot or a prosthetic foot of an individual. For instance, the shoes 210 may comprise athletic shoes, dress shoes, boots, and so forth. As shown, the shoes 210 include an insole receiving portion 212 (i.e., insole receiving portion 212A and insole receiving portion 212B) for receiving the insoles 220 (i.e., the left insole 220A and the right insole 220B), as well as soles 214 (i.e., sole 214A and sole 214B).

Accordingly, the insoles 220 can be sized and shaped to fit within the shoes 210. In an example, the insoles 220 may comprise an elastomeric and/or polymeric material such as rubberized silicone. Additionally, as illustrated, the soles 220 may include a number of components 230 (i.e., components 230A and components 230B) that are used in determining whether a turn is made. More specifically, the components 230 may include various combinations of accelerometers 232 (i.e., accelerometer 232A and accelerometer 232B), gyroscopes 234 (i.e., gyroscope 234A and gyroscope 234B), magnetometers 236 (i.e., magnetometer 236A and magnetometer 236B), global positioning system (GPS) devices 238 (i.e., GPS 238A and GPS 238B), force sensors 240 (i.e., force sensor 240A and force sensor 240B, Reed switches 242 (i.e., Reed switches 242A and Reed switches 242B), Hall Effect sensors 244 (i.e., Hall Effect sensors 244A and Hall Effect sensors 244B), tilt/incline sensors 246 (i.e., tilt/incline sensors 246A and tilt/incline sensors 246B), and communications engines 248 (i.e., communications engine 248A and communications engine 248B). Regardless of the specific combination, the components 230 may, along with the turn analysis computer system 250, be configured to determine whether an individual has turned during ambulation, as further described herein.

Additionally, each of the components 230 may comprise essentially any type of component known by one of skill in the art. For instance, any particular type of accelerometers 232 (e.g., one-axis accelerometers, two-axis accelerometers, three-axis accelerometers, and so forth), gyroscopes 234 (e.g., one-axis gyroscopes, two-axis gyroscopes, three-axis gyroscopes, and so forth), magnetometers 236 (e.g., one-axis magnetometers, two-axis magnetometers, three-axis magnetometers, and so forth), any type of appropriate GPS device, and so forth may be used to practice the principles described herein.

The communications engines 248 may comprise any combination of hardware and/or software that are configured to communicate with the communications engine 252 of the turn analysis computer system 250. In an example, the communications engines 248 may include a wireless transmitter and receiver that are configured to wirelessly communicate with the communications engine 252.

Notably, while the components 230 are shown as being positioned within the insoles 220 (and ultimately the shoes 210), the principles described herein may be practiced by utilizing many different components. For instance, some configurations may include more, and/or different, components than those illustrated as the being included within the components 230 (e.g., bilateral force sensors, strain gages, pedometers, levels, and so forth), while other configurations may include less and/or different components than those illustrated and discussed herein. In some embodiments, the components 230 can be configured to be positioned within commercially available shoes already obtained by a user, such as with a sensor insert or the insole 220. Alternatively, the components 230 can be provided in a custom shoe or foot-borne device specifically made for the components/sensors and/or the particular user. Additionally, or alternatively, the insoles 220 can replace original insoles of the shoes 210.

Furthermore, while the various components 230 are illustrated in FIG. 2 as being included within or on the insoles 220, the components 230 may be included in any location with respect to the shoes 210 or a foot of an individual. For instance, in some embodiments, one or more of the components 230 may be located within a sole 214 (e.g., sole 214A and sole 214B) of the shoes 210, located within in a lining (not shown) of the shoes 210, coupled to laces of the shoes 210, coupled to an outer portion of the shoes 210, coupled to and/or located within a sock of an individual, and so forth. Additionally, while all the components 230 are shown as being located in a single location (i.e., the insoles 220), in some embodiments, one or more first components may be positioned at a first location (e.g., the sole 214) of the shoes 220, while one or more second components are positioned at one or more second locations (e.g., insole 220, laces, socks, and so forth).

As briefly discussed, the environment 200 also includes the turn analysis computer system 250. The turn analysis computer system may correspond to the computer system 100, as described with respect to FIG. 1. The turn analysis computer system 250 may comprise any type of computer system that is configured to aid in determining whether an individual has made a turn during ambulation, as further described herein. In an example, the turn analysis computer system 250 may comprise a desktop computer, a laptop computer, a tablet, a smartphone, and so forth. Furthermore, the free hand conversion computer system 210 may be running any applicable operating system, including but not limited to, MICROSOFT® WINDOWS®, APPLE® MACOS®, APPLE IOS GOOGLE™ CHROME OS™ ANDROID™, LINUX®, UBUNTU®, and so forth.

As shown, the turn analysis computer system 250 may include various engines, functional blocks, and components, including communications engine 252, timing engine 254, turn data analytics engine 256, and turn user interface 258. The various engines, components, and/or functional blocks of the turn analysis computer system 250 may be implemented on a local computer system or may be implemented on a distributed computer system that includes elements resident in the cloud or that implement aspects of cloud computing (i.e., at least one of the various illustrated engines may be implemented locally, while at least one other engine may be implemented remotely).

Alternatively, or additionally, one or more of the engines of the turn analysis computer system may be located remote from the components 230 and/or shoes 210, such as on a hip or belt worn housing. Alternatively, or additionally, one or more of the engines of the turn analysis computer system may be located in or on the shoe, or around an ankle or prosthesis of an individual. Alternatively, or additionally, one or more of the engines of the turn analysis computer system may be formed together on a circuit within the shoe or insole. Alternatively, or additionally, one or more of the engines of the turn analysis computer system may be located in a wrist-worn device or in a housing that can be put in a pocket of a clothing item of an individual.

While the illustrated engines (i.e., the communications engine 252, the timing engine 254, the turn data analytics engine 256, and the turn user interface 258) of the turn analysis computer system 250 are shown as being part of a separate computer system (i.e., the turn analysis computer system 250), one or more of the illustrated engines may be included as part of the footwear (e.g., within the shoes 210, within or coupled to the insoles 220, coupled to socks, coupled to laces, and so forth) of an individual, as further described herein with respect to the components 230. The various engines, functional blocks, and/or components of the free hand conversion computer system 210 may be implemented as software, hardware, or a combination of software and hardware.

Notably, the configuration of turn analysis computer system 250 (and the components 230) illustrated in FIG. 2 is shown only for exemplary purposes. As such, the turn analysis computer system 250 (and the components 230) may include more or less than the engines, functional blocks, and/or components illustrated in FIG. 2. Although not illustrated, the various engines of the turn analysis engine computer system 250 (and the components 230) may access and/or utilize a processor and memory, such as the processor 102 and the memory 104 of FIG. 1, as needed to perform their various functions.

As briefly described, the turn analysis computer system includes the communications engine 252, the timing engine 254, the turn data analytics engine 256, and the turn user interface 258. The communications engine 252 may comprise any combination of hardware and/or software that is configured to communicate with the communications engine 240 of the components 230. The timing engine 254 may be responsible for determining a relative start time that is used for determining stances or strides for one or both a left foot and right foot of an individual (and ultimately, for determining whether an individual has made a turn during ambulation).

In some embodiments, the timing engine may include a real-time clock at the footwear of an individual (e.g., the shoes 210, the insoles 230, laces, socks, and so forth), which real-time clocks may then be synced with respect to one another (i.e., a left foot clock with a right foot clock) for determining a relative time at which each stance or stride has occurred. In other embodiments, the timing engine may include any appropriate hardware and/or software at the footwear of an individual (e.g., the shoes 210, the insoles 230, laces, socks, and so forth) for communicating with each other such that a relative time may be determined (e.g., an agreement that a certain time is to be considered time zero as a reference point for determining a point in time associated with each identified stance or stride).

The turn data analytics engine 256 may be configured to analyze the data (e.g., stance and/or stride data) gathered by the components 230 as described herein. In an example, the turn data analytics engine may comprise a computer, at least a portion of a hardware and/or software processor of a computer, a microprocessor, a microcontroller, and so forth.

Finally, the turn user interface 258 may comprise any appropriate user interface (e.g., a user interface associated with an application) that allows for presenting information associated with data gathered and analyzed by the components 230 and the various engines of the turn analysis computer system 250. For instance, an application including the turn user interface 258 may allow an individual to view foot parameters, stance data, stride data, and so forth associated with a particular user of footwear as shown and described with respect to the environment 200 of FIG. 2.

Before proceeding further, it may be useful to provide definitions for certain terms used herein. For instance, a “stance” is the duration in time that a foot is in contact with the floor or ground. A “stride” is the movement of one foot from one stance to a following stance. Furthermore, a “foot parameter” may be substantially any characteristic of a foot (e.g., orientation, weight distribution, etc.) that can be detected or determined from the components 230 alone or in combination with the turn analysis computer system 250.

Reference herein to a “first foot parameter” refers to a particular parameter, not necessarily a particular foot. For instance, a first foot parameter may refer to a particular parameter of a left foot, a right foot, or both feet. Similarly, reference herein to a “second foot parameter” refers to a particular parameter, not necessarily a particular foot. For instance, a second foot parameter may refer to a particular parameter of a left foot, a right foot, or both feet. In some embodiments, first and second foot parameters are identified for a single foot (e.g., a left foot or a right foot). In other embodiments, a first foot parameter may be identified for one foot (e.g., a left foot) and a second foot parameter may be identified for another foot (e.g., a right foot). In still other embodiments, first and second foot parameters may be identified for both a left foot and a right foot.

Moreover, a frame of reference (generally or for each foot) is also referenced herein. The frame of reference may include x-, y-, and z-axes. The x-axis may extend along or parallel to the long edge of an individual's foot, the y-axis may extend along the short edge of the individual's foot, and the z-axis may extend perpendicular to the individual's foot.

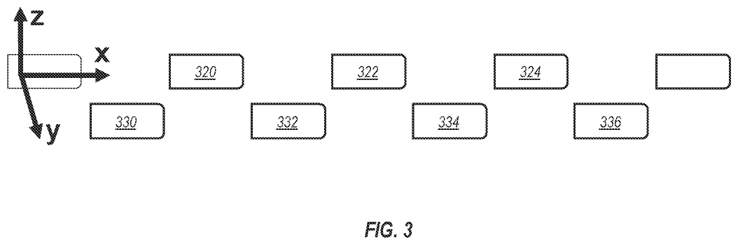

As briefly discussed, the components 230 may aid the turn analysis computer system 250 in determining whether an individual has turned during ambulation. In particular, the components 230 may be configured to work in combination to determine when an individual has turned during ambulation, as further described with respect to FIGS. 3-5.

As shown, FIG. 3 illustrates a series of left stances (i.e., left stance 320, left stance 322, and left stance 324) and a series of right stances (i.e., right stance 330, right stance 332, right stance 334, and right stance 336) that occur while an individual ambulates. Parameters of the left foot and/or the right foot during the stances and/or strides can be determined based on the signals or data from the components 230. Changes in the foot parameters can be used to determine if an individual has made a turn during ambulation.

By way of example, a first foot parameter can be determined for each of stance 330, stance 332, and stance 334. For instance, the magnetometer 236B may detect data regarding the right foot in the xy plane during each of stance 330, stance 332, and stance 334. The magnetometer data for each of stance 330, stance 332, and stance 334 may then be compared to each other to determine whether there is a change in the data from one stance to another.

Similarly, a second foot parameter can be determined between each of stance 330, stance 332, stance 334, and stance 336 (i.e., during the strides between stance 330 and stance 332, between stance 332 and stance 334, and between stance 334 and stance 336). For instance, the gyroscope 264B may detect data regarding the right foot about the z-axis between stance 330, stance 332, stance 334, and stance 336. The gyroscope data between stance 330, stance 332, stance 334, and stance 336 may then be compared to each other to determine whether there is a change in the data from one stride to another.

Sufficient changes detected in the first and second foot parameters can be used to determine that the individual has made a turn during ambulation. In some embodiments, the changes in the first and second foot parameters must meet or exceed predetermined threshold change levels in order to be considered in a determination of whether a turn has been made. If the changes in the first and second foot parameters are not large enough to meet or exceed the predetermined threshold change levels, it may be presumed that the changes are indicative of normal changes that occur while an individual ambulates in a straight line. In some embodiments, the predetermined threshold change levels may be differences of 50% or more, 50% or less, 25%, 20%, 15%, 10%, 5%, 2.5%, less than 2.5%, or between any of the forgoing values.

In some embodiments, the predetermined threshold change levels may vary between the first foot parameter and the second foot parameter. For instance, the predetermined threshold change level for the first foot parameter may be 10% while the predetermined threshold change level for the second foot parameter may be 25%. In any event, if the changes in the first foot parameter and/or the second foot parameter meet or exceed the predetermined threshold change level, the data from the components 230 may be used to determine if a turn has been made.

FIG. 3 is described in the context of comparing changes in the first and second foot parameters of just the individual's right foot. As noted above, a similar analysis may be performed while comparing changes in a first foot parameter of one foot (e.g., a left foot) and changes in a second foot parameter of the other foot (e.g., a right foot). By way of example, a first foot parameter can be determined for each of stance 320, stance 322, and stance 324 of the left foot and a second foot parameter can be determined during the strides between stance 330, stance 332, stance 334, and stance 336 of the right foot, or vice versa. In other embodiments, the analysis may be done by only looking at the left foot (e.g., identifying changes in a first foot parameter from stance 320 to stance 322 to stance 324 and identifying changes in a second foot parameter during the strides between stance 320, stance 322, and stance 324). In still other embodiments, additional foot parameters (e.g., third foot parameter, fourth foot parameter, etc.) may also be identified and analyzed for one or both of the individual's feet in order to determine whether the individual has turned during ambulation.

As illustrated in FIG. 3, the individual is ambulating in a straight line. This would be identified using the components 230 because the changes in the first and second foot parameters would be below the predetermined threshold change levels. As noted, this could be determined regardless of whether the first and second foot parameters are collected from one or both of the individual's feet.

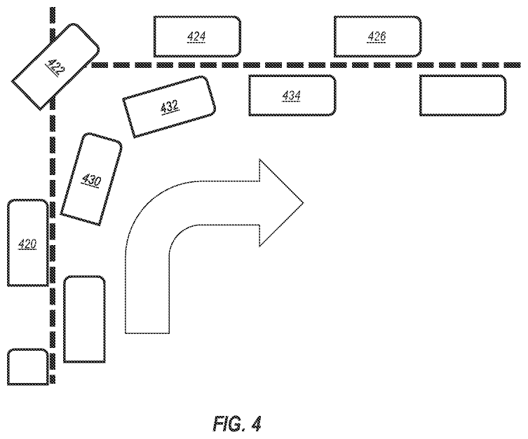

Attention is now directed to FIG. 4, which illustrates a series of left stances (i.e., left stance 420, left stance 422, left stance 424, and left stance 426) and a series of right stances (i.e., right stance 430, right stance 432, and right stance 434) that occur while an individual ambulates. As can be seen in FIG. 4, the illustrated stances show that the individual has made a right turn of about 90° between stance 420 and stance 426. The systems and methods described herein can be used to determine that the turn has been made. For instance, parameters of the left foot and/or the right foot during the stances and/or strides can be determined based on the signals or data from the components 230. Changes in the foot parameters can be used to determine that the individual has made the turn.

By way of example, a first foot parameter can be determined for the left foot and/or the right foot. For instance, the magnetometer 236A may detect data regarding the left foot in the xy plane during each of stance 420, stance 422, stance 424, and stance 426. Additionally or alternatively, the magnetometer 236B may detect data regarding the right foot in the xy plane during each of stance 430, stance 432, and stance 434. The magnetometer data for one or both sets of stances (e.g., stances 420, 422, 424, 426 and/or stances 430, 432, 434) may then be compared to determine whether there is a change in the data from one stance to another.

Similarly, a second foot parameter can be determined for the left foot and/or the right foot. For instance, the gyroscope 264A may detect data regarding the left foot about the z-axis during the strides between stance 420, stance 422, stance 424, and stance 426. Additionally or alternatively, the gyroscope 264B may detect data regarding the right foot about the z-axis during the strides between stance 430, stance 432, and stance 434. The gyroscope data for one or both sets of strides (e.g., strides between stances 420, 422, 424, 426 and/or strides between stances 430, 432, 434) may then be compared to determine whether there is a change in the data from one stride to another.

As noted above, sufficient changes detected in the first and second foot parameters can be used to determine that the individual has made a turn during ambulation. For instance, changes in the first and second foot parameters that meet or exceed predetermined threshold change levels may indicate that a turn has been made.

Furthermore, the specific changes in the first and/or second foot parameters may also indicate the direction of the turn (e.g., to the right or to the left). For instance, the components 230 may detect data about the stances and/or strides that indicate the individual has made a right turn or a left turn. Such data may be reflective of a weight shift in a particular direction, orientation of one or both feet in a particular direction, and the like.

Moreover, the magnitude or extent of the changes in the first and/or second foot parameters can also be used to identify or estimate the sharpness or angle of the turn. For instance, in some embodiments, larger changes in the first and/or second foot parameters may indicate a sharper turn, while smaller changes may indicate a more milder turn. By way of example, the components 230 may detect data about the stances and/or strides that are indicative of the magnitude of the first and/or second foot parameters. For instance, in some embodiments, the components 230 may detect the magnitude of a weight shift in a particular direction, orientation of one or both feet in a particular direction, and the like. This data can be used to determine or approximate the angle of the turn.

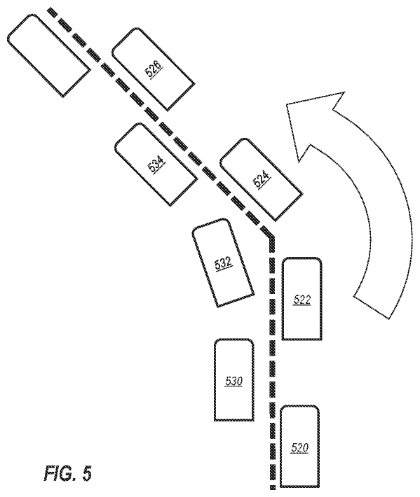

Attention is now directed to FIG. 5, which illustrates a series of right stances (i.e., right stance 520, right stance 522, right stance 524, and right stance 526) and a series of left stances (i.e., left stance 530, left stance 532, and left stance 534) that occur while an individual ambulates. As can be seen in FIG. 5, the illustrated stances show that the individual has made a left turn of about 45° between stance 520 and stance 526.

As with the previous embodiments, parameters of the left foot and/or the right foot during the stances and/or strides can be determined based on the signals or data from the components 230. Changes in the foot parameters can be used to determine that the individual has made the turn and the extend or degree of the turn.

The embodiments of FIG. 3-5 illustrate various ambulation patterns (e.g., straight, right turn, and left turn) using a “7 stance window.” More specifically, the noted example use data from seven stances and/or strides between the seven stances. It will be appreciated that using a 7 stance window is merely exemplary. In other embodiments, a stance window of fewer or more than second stances could be used. In some embodiments, using a stance window of fewer or more than seven stances may allow for or require adjustments to the threshold change levels discussed herein.

In any event, determining whether an individual has made a turn may be performed (e.g., by the turn data analytics engine 256) in real-time (i e, immediately, or almost immediately, upon gathering turn data). In other embodiments, such determinations may be performed at a later time.

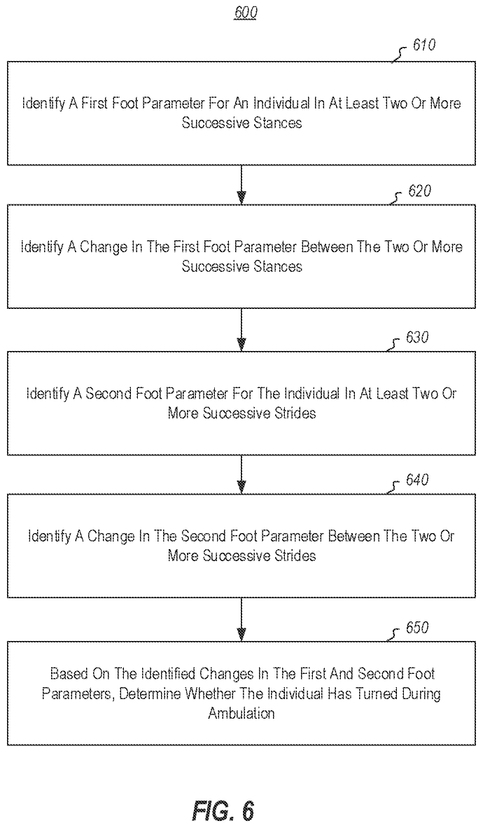

FIG. 6 illustrates a flowchart of a method 600 for determining a whether an individual has made a turn during ambulation. The method 600 is described with frequent reference to the environments of FIGS. 2-5.

The method 600 includes identifying a first foot parameter for an individual in at least two or more successive stances (step 610). For example, a first foot parameter may be identified for the right foot from FIG. 4. In one embodiment, magnetometer 236B may detect information about the right foot in the xy plane during two or more successive stances of stances 430, 432, 434.

The method 600 also includes identifying a change in the first foot parameter between the two or more successive stances (step 620). For instance, the difference in the first foot parameter between stance 430 and stance 432 and/or between stance 432 and stance 434 from FIG. 3 can be determined.

The method 600 further includes identifying a second foot parameter for an individual in at least two or more successive strides (step 630). For example, a second foot parameter may be identified for the left foot from FIG. 4. In one embodiment, gyroscope 234A may detect information about the left foot about the z-axis during two or more successive strides (e.g., strides between stances 420, 422, stances 422, 424, and/or stances 424, 426).

The method 600 also includes identifying a change in the second foot parameter between the two or more successive strides (step 640). For instance, the difference in the second foot parameter between successive strides (e.g., strides between stances 420, 422, stances 422, 424, and/or stances 424, 426) can be determined.

The method also includes determining whether the individual has turned during ambulation, based on the identified changes in the first and second foot parameters (step 650). A determination of whether an individual has turned during ambulation can be made by determining whether the changes in one or both of the first and second foot parameters meets or exceeds one or more predetermined threshold change levels. Changes in one or both of the first and second foot parameters that meets or exceeds the one or more predetermined threshold change levels indicate that the individual has turned during ambulation.

The method 600 may also include additional steps to determine the direction of the turn and/or the sharpness or degree of the turn.

In this way, one or more components for determining turns during ambulation may be coupled to an individual via footwear (e.g., shoes, boots, socks, and so forth) of the individual. The one or more components and potentially, one or more components separate from the footwear, may gather and analyze data associated with stances or strides of the individual. This data may then be analyzed to determine whether the individual has made one or more turns during ambulation. Such determinations may then allow for taking preventative and/or corrective measures or improve the individual's ambulation.

Although the subject matter has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. A computer system comprising:
one or more processors; and
one or more computer-readable storage media having stored thereon computer-executable instructions that are executable by the one or more processors to cause the computer system to determine whether an individual has turned during ambulation, the computer-executable instructions including instructions that are executable to cause the computer system to perform at least the following:
identify a first foot parameter for the individual in at least two or more successive stances;
identify a change in the first foot parameter between the two or more successive stances;
identify a second foot parameter for the individual in at least two or more successive strides;
identify a change in the second foot parameter between the two or more successive strides; and
based on the identified changes in the first and second foot parameters, determine whether the individual has turned during ambulation.

2. The computer system in accordance with claim 1, wherein the first foot parameter and the second foot parameter are identified for the same foot of the individual.

3. The computer system in accordance with claim 1, wherein the first foot parameter is identified for one foot of the individual and the second foot parameter is identified for another foot of the individual.

4. The computer system in accordance with claim 1, wherein the first foot parameter is identified using one or more magnetometers.

5. The computer system in accordance with claim 1, wherein the second foot parameter is identified using one or more gyroscopes.

6. The computer system in accordance with claim 1, wherein the first foot parameter comprises data regarding a foot of the individual in an xy plane during the at least two or more successive stances.

7. The computer system in accordance with claim 1, wherein the second foot parameter comprises data regarding a foot of the individual in about a z-axis during the at least two or more successive strides.

8. A method, implemented at a computer system that includes one or more processors, for determining whether an individual has turned during ambulation, comprising:
identifying a first foot parameter for the individual in at least two or more successive stances;
identifying a change in the first foot parameter between the two or more successive stances;
identifying a second foot parameter for the individual in at least two or more successive strides;
identifying a change in the second foot parameter between the two or more successive strides; and
based on the identified changes in the first and second foot parameters, determining whether the individual has turned during ambulation.

9. The method in accordance with claim 8, wherein the at least two or more successive stances and the at least two or more successive strides are taken from a 7 stance window.

10. The method in accordance with claim 8, further comprising identifying a direction of a turn made by the individual during ambulation based on the identified changes in the first and second foot parameters.

11. The method in accordance with claim 8, further comprising identifying a degree or sharpness of a turn made by the individual during ambulation based on the identified changes in the first and second foot parameters.

12. The method in accordance with claim 8, wherein the first foot parameter and the second foot parameter are identified for the same foot of the individual.

13. The method in accordance with claim 8, wherein the first and second foot parameters are identified using at least one axis of an accelerometer, a gyroscope, or a magnetometer.

14. The method in accordance with claim 8, wherein at least one component used to identify at least one of the first foot parameter or the second foot parameter is located within footwear worn by the individual.

15. A computer program product comprising one or more computer readable media having stored thereon computer-executable instructions that are executable by one or more processors of a computer system to cause the computer system to determine whether an individual has turned during ambulation, the computer-executable instructions including instructions that are executable to cause the computer system to perform at least the following:
identify a first foot parameter for the individual in at least two or more successive stances;
identify a change in the first foot parameter between the two or more successive stances;
identify a second foot parameter for the individual in at least two or more successive strides;
identify a change in the second foot parameter between the two or more successive strides; and
based on the identified changes in the first and second foot parameters, determine whether the individual has turned during ambulation.

16. The computer program product in accordance with claim 15, wherein the first foot parameter is identified for one foot of the individual and the second foot parameter is identified for another foot of the individual.

17. The computer program product in accordance with claim 15, further comprising identify a third foot parameter for the individual in the at least two or more successive stances or the at least two or more successive strides.

18. The computer program product in accordance with claim 17, further comprising identify a change in the third foot parameter between the two or more successive stances or the at least two or more successive strides.

19. The computer program product in accordance with claim 15, wherein the first foot parameter and the second foot parameter are identified using one or more of an accelerometer, a gyroscope, a magnetometer, and a GPS device.

20. The computer program product in accordance with claim 15, wherein the determination of whether the individual has turned during ambulation is made substantially in real-time.
